(19) United States
(12) Patent Application Publication
(10) Pub. No.: US 2005/0004179 A1
(43) Pub. Date: Jan. 6, 2005

(54) Methods and materials for treating, detecting, and reducing the risk of developing Alzheimer's Disease

(76) Inventor: Ward A. Pedersen, Bellevue, NE (US)

Correspondence Address: Rogalskyj & Weyand, LLP, P.O. Box 44, Livonia, NY, 14487-0044, US

(21) Appl. No.: 10/851,508

(22) Filed: May 22, 2004

Related U.S. Application Data
(60) Provisional application No. 60/472,558, filed on May 22, 2003.
(60) Provisional application No. 60/544,638, filed on Feb. 13, 2004.

(51) Int. Cl. A61K 31/4439; A61K 31/426

(52) U.S. Cl. 514/342; 514/369

(57) ABSTRACT

Disclosed are methods for treating Alzheimer's Disease in a subject and for preventing or inhibiting the development of Alzheimer's Disease in a subject. The methods include administering, to the subject, a material which decreases insulin resistance. Also disclosed is a composition which includes a material that decreases insulin resistance and an antidepressant/anti-anxiety drug. Also disclosed are articles of manufacture which include a container; a material that decreases insulin resistance disposed within the container; and a label affixed to the container and/or an insert disposed in the container, where the label and/or insert indicates and/or suggests that the material can be used to treat, decrease the risk for development of, and/or inhibit the development of Alzheimer's Disease and/or one or more clinical features of Alzheimer's Disease.

The present application claims the benefit of U.S. Provisional Patent Application Ser. No. 60/472,558, filed May 22, 2003 and of U.S. Provisional Patent Application Ser. No. 60/544,638, filed Feb. 13, 2004, which provisional patent applications are hereby incorporated by reference.

### FIELD OF THE INVENTION

The subject invention is directed, generally, to methods for the detection, treatment, and/or prevention of diseases and conditions of the brain and, more particularly, to methods for the detection, treatment, and/or prevention of Alzheimer's Disease and/or one or more clinical features of Alzheimer's Disease.

### BACKGROUND OF THE INVENTION

Alzheimer's Disease (“AD”) is a major cause of dementia among the elderly throughout the world. Beginning at age 65, the incidence of the disease rises steadily until, by age 85, conservative estimates place its rate of incidence at some 30% of that population. It is generally believed that the disease begins a number of years before it manifests itself in the mild cognitive changes that are the early signs of AD. Thus, the at risk population is believed to be 60 years or older.

The consequences of this disease are devastating, both to the patient and his or her family and care givers. The disease typically results in an inexorable decline in cognitive functions and impairments in short-term memory function. These are frequently accompanied by behavioral problems consistent with depression and anxiety, such as irritability, severe restlessness and hyperactivity, and poor sleep patterns. As a result, the disease leads to the patient's inability to care for him or herself in the community and places increased burdens on care givers and home care and nursing home providers.

The high prevalence of AD, combined with the rate of growth of the elderly segment of the population, make this disease one of the most important current public health problems. For at least this reason, a need continues for methods of treating AD, and the present invention is directed, in part, to addressing this need.

### SUMMARY OF THE INVENTION

The present invention relates to a method for treating Alzheimer's Disease in a subject. The method includes administering, to the subject, a material which decreases insulin resistance.

The present invention also relates to a method for preventing or inhibiting the development of Alzheimer's Disease in a subject. The method includes administering, to the subject, a material which decreases insulin resistance.

The present invention also relates to a composition which includes a material which decreases insulin resistance and an antidepressant/anti-anxiety drug.

The present invention also relates to an article of manufacture. The article of manufacture includes a container; a material which decreases insulin resistance disposed within the container; and a label affixed to the container and/or an insert disposed in the container, wherein the label and/or insert indicates and/or suggests that the material can be used to treat, decrease the risk for development of, and/or inhibit the development of Alzheimer's Disease and/or one or more clinical features of Alzheimer's Disease.

The present invention also relates to a method for assessing a test compound's ability to decrease insulin resistance. The method includes administering the test compound to a Tg2576 mouse and evaluating whether the Tg2576 mouse exhibits a decrease in insulin resistance.

The present invention also relates to a method for detecting Alzheimer's Disease in a subject or for assessing a subject's risk for developing Alzheimer's Disease. The method includes assessing insulin resistance in the subject, a high level of insulin resistance being indicative of the presence of Alzheimer's Disease or of an increased risk for developing Alzheimer's Disease.

The present invention also relates to a method for assessing a subject's risk for developing memory impairment and/or abnormal stress responses. The method includes assessing insulin resistance in the subject, a high level of insulin resistance being indicative of an increased risk for developing memory impairment and/or abnormal stress responses.

The present invention also relates to a method for improving spatial learning or for inhibiting spatial learning deficits in a subject. The method includes administering, to the subject, a material which decreases insulin resistance.

The present invention also relates to a method for reducing or inhibiting the development of memory impairment or for improving memory in a subject. The method includes administering, to the subject, a material which decreases insulin resistance.

### BRIEF DESCRIPTION OF THE DRAWINGS

FIGS. 1A and 1B are graphs showing insulin-induced decline in blood glucose levels for 5-month-old Tg2576 and wild-type mice (FIG. 1A) and for 8-month-old Tg2576 and wild-type mice (FIG. 1B). Non-fasted mice were administered insulin at 0.75 U/kg and the levels of blood glucose were determined at 0, 10, 30, and 60 min following injection. Data are expressed as a percent of the control values (0 time point determinations). Values are means and SEMs of determinations made in 7 Tg2576 mice and 7 wild-type mice (*p<0.04; unpaired t test).

FIGS. 2A and 2B are bar graphs showing normal glycemic control in Tg2576 mice. The basal concentration of blood glucose (FIG. 2A) and percent glycosylated hemoglobin (%HbA1c) (FIG. 2B) were determined in 8-month-old, non-fasted Tg2576 and wild-type mice. The differences in either measure were not statistically different between the two groups of mice. Values are means and SEMs of determinations made in the same animals used for the insulin response study (FIGS. 1A and 1B).

FIGS. 3A, 3B, and 3C are bar graphs showing differential regulation of insulin and corticosterone levels in Tg2576 mice. FIG. 3A shows that, under non-fasting conditions, there was no difference between the levels of serum insulin between wild-type (Wt) and Tg2576 (Tr) mice at 5 months of age (5 m), but the levels were lower in Tr mice at 8 months of age (8 m) (*p<0.04 vs. Tr, 5 m by paired t test; **p<0.03 vs. Wt, 8 m by unpaired t test). FIG. 3B shows that, in fasted Tr mice, serum insulin levels were undetectable in all animals at both ages examined, whereas only two fasted Wt mice at 5 m and only one fasted Wt mouse at 8 m had undetectable serum insulin levels. FIG. 3C shows that, at 8 m, there was no statistically significant difference between the serum corticosterone levels of non-fasted (NF) Tr and Wt mice, but the levels were higher in Tr mice than in Wt mice when fasted overnight (F) (***p<0.02 vs. Wt, F; unpaired t test). Similar results were obtained for mice at 5 m. Values are means and SEMs of determinations made in the same animals used for the insulin response study (FIGS. 1A and 1B).

FIGS. 4A and 4B are graphs showing that rosiglitazone normalizes the insulin response of Tg2576 mice. Non-fasted mice were administered insulin at 0.75 U/kg and the levels of blood glucose were determined at 0, 10, 30, and 60 min following injection. Data are expressed as a percent of the control values (0 time point determinations). FIG. 4A shows the insulin-induced decline in blood glucose levels for 10-month-old wild-type mice fed a regular diet and 10-month-old Tg2576 mice fed the rosiglitazone-supplemented diet for 4 weeks (Tr, AV). Values are means and SEMs of determinations made in 7 wild-type and 7 Tr, AV mice. FIG. 4A shows the insulin-induced decline in blood glucose levels for 10-month-old Tg2576 mice fed a regular diet (Tr, Cont) and 10-month-old Tg2576 mice fed the rosiglitazone-supplemented diet for 4 weeks (Tr, AV). Values are means and SEMs of determinations made in 5 Tr, Cont and 7 Tr, AV mice (*p<0.05 vs Tr, AV at 10 min; **p<0.03 vs Tr, AV at 30 min; unpaired t test).

FIG. 5 is a bar graph showing that rosiglitazone normalizes the stress response of Tg2576 mice. Mice were fasted overnight, and serum corticosterone concentrations were determined. The levels were not significantly different between 10-month-old wild-type mice (Wt) and 10-month-old Tg2576 mice fed the rosiglitazone-supplemented diet for 4 weeks (Tr, AV), but there was a statistically significant difference between the levels of 10-month-old Tg2576 mice fed a regular diet (Tr, Cont) and the other two groups (*p<0.05 vs Wt or Tr, AV; one-way ANOVA and Tukey post hoc test). Values are means and SEMs of determinations made in 7 Wt, 7 Tr, AV and 5 Tr, Cont mice.

FIG. 6 is a bar graph demonstrating that increasing insulin sensitivity in a transgenic mouse model of Alzheimer's Disease reverses the age-dependent decline in memory performance of the animals.

### DETAILED DESCRIPTION OF THE INVENTION

The present invention, in one aspect thereof, relates to a method for treating Alzheimer's Disease in a subject. The method includes administering, to the subject, a material which decreases insulin resistance.

The present invention, in another aspect thereof, relates to a method for preventing or inhibiting the development of Alzheimer's Disease in a subject. The method includes administering, to the subject, a material which decreases insulin resistance.

“Subject”, as used herein is meant to include mammals, such as humans, other primates, dogs, rats, and mice. Illustratively, the subject can be a human or other subject that suffers from HPA axis dysfunction, or the subject can be a human or other subject that does not suffer from HPA axis dysfunction; the subject can be a human or other subject that suffers from spatial learning deficits, subject can be a human or other subject that does not suffer from spatial learning deficits; the subject can be a human or other subject that exhibits increased amyloid β-peptide levels, the subject can be a human or other subject that does not exhibit increased amyloid β-peptide levels; the subject can be a human or other subject that suffers from type II diabetes, or the subject can be a human or other subject that does not suffer from type II diabetes; the subject can be a human or other subject that suffers from hyperlipidaemia, or the subject can be a human or other subject that does not suffer from hyperlipidaemia; the subject can be a human or other subject that suffers from hypertension, or the subject can be a human or other subject that does not suffer from hypertension; the subject can be a human or other subject that suffers from cardiovascular disease, or the subject can be a human or other subject that does not suffer from cardiovascular disease; and/or the subject can be a human or other subject that suffers from an eating disorder (such as anorexia nervosa, obesity, and anorexia bulimia), or the subject can be a human or other subject that does not suffer from an eating disorder (such as anorexia nervosa, obesity, and anorexia bulimia).

As used herein, “a material which decreases insulin resistance” refers to any material (i) which has been shown or can be shown to decrease insulin resistance and/or (ii) which decreases insulin resistance in the subject. As one skilled in the art will appreciate, insulin resistance can be assessed by a variety of methods, such as a glucose tolerance test, and a variety of materials can be used to decrease insulin resistance. Illustratively, materials which decrease insulin resistance include those which increase the body's sensitivity and response to insulin (the so-called “insulin sensitizers”), such as those which decrease insulin resistance by improving insulin sensitivity in peripheral tissues.

Illustratively, the material can be a compound described in U.S. Pat. No. 5,002,953 to Hindley, which is hereby incorporated by reference, such as a compound having a formula set forth and described at column 1, line 35 to column 4, line 33 of U.S. Pat. No. 5,002,953 to Hindley, which is hereby incorporated by reference, and/or such as one of the compounds listed in claim 12 of U.S. Pat. No. 5,002,953 to Hindley, which is hereby incorporated by reference.

Still illustratively, the material can be a compound described in U.S. Pat. No. 5,965,584 to Ikeda et al., which is hereby incorporated by reference, such as a compound having Formula I, II, and/or III as set forth and/or described at column 2, lines 15-36, at column 2, line 50 to column 3, line 4, and/or at column 3, line 15 and/or as further described at column 3, line 45 to column 10, line 54 of U.S. Pat. No. 5,965,584 to Ikeda et al., which is hereby incorporated by reference.

Still illustratively, the material can be a compound described in U.S. Pat. No. 4,687,777 to Meguro et al., which is hereby incorporated by reference, such as a compound having Formula I as set forth and/or described at column 1, lines 41-51 and/or at column 2, lines 1-23 of U.S. Pat. No. 4,687,777 to Meguro et al., which is hereby incorporated by reference.

Still illustratively, the material can be a compound described in U.S. Pat. No. 4,572,912 to Yoshioka et al., which is hereby incorporated by reference, such as a compound having Formula Ia, Ib, and/or Ic as set forth and/or described at column 2, line 56 to column 15, line 3 of U.S. Pat. No. 4,572,912 to Yoshioka et al., which is hereby incorporated by reference.

In one illustrative embodiment, the present invention can be practiced using an agonist of peroxisome proliferator-activated receptor-γ (“PPARγ”) which decreases insulin resistance.

In another illustrative embodiment, the present invention can be practiced using a thiazolidinedione material which decreases insulin resistance, such as in the case where the thiazolidinedione material which decreases insulin resistance is a glitazone or a pharmaceutically acceptable salt, solvate, or adduct thereof. Examples of such glitazones or pharmaceutically acceptable salts, solvates, or adducts thereof include rosiglitazones or pharmaceutically acceptable salts, solvates, or adducts thereof, one example of which is rosiglitazone maleate; pioglitazones pharmaceutically acceptable salts, solvates, or adducts thereof, examples of which include pioglitazone hydrochloride and pioglitazone maleate; troglitazone or pharmaceutically acceptable salts, solvates, or adducts thereof; englitazone or pharmaceutically acceptable salts, solvates, or adducts thereof; ciglitazone or pharmaceutically acceptable salts, solvates, or adducts thereof; and darglitazone or pharmaceutically acceptable salts, solvates, or adducts thereof. Some of these compounds are commercially available, or they can be prepared using known methods, such as those described in U.S. Pat. No. 5,002,953 to Hindley; U.S. Pat. No. 4,687,777 to Meguro et al.; U.S. Pat. No. 5,965,584 to Ikeda et al.; and U.S. Pat. No. 4,572,912 to Yoshioka et al., each of which is hereby incorporated by reference. Other suitable materials which can be used in the practice of the present invention include those described in U.S. Pat. No. 5,741,803 to Pool et al.; U.S. Pat. No. 6,166,042 to Ikeda et al.; and/or U.S. Pat. No. 6,288,095 to Hindley et al., each of which is hereby incorporated by reference.

Other materials which decrease insulin resistance and, thus, which can be used in the practice of the methods of the present invention can be identified and/or evaluated, for example, using any suitable screening method. For example, in one such screening method, to which the present invention, in still another aspect thereof, relates, a test compound's ability to decrease insulin resistance is assessed by administering the test compound to a Tg2576 mouse and evaluating whether the Tg2576 mouse exhibits a decrease in insulin resistance, for example, by monitoring blood glucose concentrations. The Tg2576 mouse is commercially available and can be produced, for example, as indicated in U.S. Pat. No. 5,877,399 to Hsiao, which is hereby incorporated by reference. Administration of the test compound to the Tg2576 mouse can be carried out using any suitable route or formulation, such as the routes and formulations described hereinbelow. The screening method can be carried out with the Tg2576 mouse in a stressed state or in an unstressed state, or the screening method can be carried out with a plurality of mice, some of which are stressed and others of which are unstressed. The screening method can involve other, additional steps. For example, the screening method can further involve assessing the test compound's effect on one or more symptoms of Alzheimer's Disease or on Alzheimer's Disease itself. Additionally or alternatively, the screening method can involve further steps, such as administering the test compound to a human, for example, to further evaluate its ability to decrease insulin resistance and/or to assess its ability to treat Alzheimer's Disease, such as by assessing its ability to treat a clinical manifestation of Alzheimer's Disease, such as memory impairment.

As discussed above, the aforementioned materials are useful in treating and/or inhibiting the development of Alzheimer's Disease in subjects, such as humans, other primates, dogs, rats, mice, and other mammals, for example, in a therapeutically or prophylactically effective amount. As used herein, “treating Alzheimer's Disease” is meant to include any qualitatively or quantitatively observable or measurable prevention, inhibition, or reversal of the progress of Alzheimer's Disease or any one or more clinical manifestations of the disease, such as any qualitatively or quantitatively observable or measurable prevention, inhibition, or reversal in any one or more of the following: memory impairment, spatial learning deficits, depression, abnormal anxiety levels, and abnormal stress responses. As used herein, “preventing or inhibiting the development of Alzheimer's Disease” is meant to include any qualitatively or quantitatively observable or measurable prevention or delay in the onset of Alzheimer's Disease or any one or more clinical manifestations of the disease, such as any qualitatively or quantitatively observable or measurable prevention, inhibition, or reversal in any one or more of the following: memory impairment, spatial learning deficits, depression, abnormal anxiety levels, and abnormal stress responses. As used herein, the term “prophylactically effective amount” is the quantity of glitazone or other material which decreases insulin resistance required to prevent or delay the onset of Alzheimer's Disease in a mammal or other subject susceptible (by reason of age, genetic disposition, family history, etc.) to contracting Alzheimer's Disease; and, as used herein, the term “therapeutically effective amount” is the quantity of glitazone or other material which decreases insulin resistance sufficient to prevent, retard, or reverse the progress of Alzheimer's Disease in a mammal or other subject already afflicted with Alzheimer's Disease.

The method of the present invention can further include administering one or more antidepressant/anti-anxiety drugs. As used herein, “antidepressant/anti-anxiety drugs” refer to drugs which reduce or inhibit depression and/or anxiety levels or other responses to stress. Suitable antidepressant/anti-anxiety drugs for use in such formulations include selective serotonin reuptake inhibitors (“SSRIs”), such as citalopram (e.g., Celexa™), escitalopram (e.g., Lexapro™), sertraline (e.g., Zoloft™), paroxetine (e.g., Paxil™) and fluoxetine (e.g., Prozac™). Other suitable antidepressant/anti-anxiety drugs that can be used in such formulations include those which inhibit reuptake of brain chemicals other than serotonin, such as venlafaxine (e.g., Effexor™), mirtazapine (e.g., Remeron™) and bupropion (e.g., Wellbutrin™). Still other suitable antidepressant/anti-anxiety drugs that can be used in such formulations include tricyclic antidepressants, such as nortriptyline (e.g., Pamelor™) and desipramine (e.g., Norpramine™). Still other suitable antidepressant/anti-anxiety drugs that can be used in such formulations include anti-anxiety drugs such as buspirone (e.g., Buspa™) and clonazepam (e.g., Klonopin™). The glitazone or other material which decreases insulin resistance and the antidepressant/anti-anxiety drug can be administered simultaneously or non-simultaneously. Simultaneous administration is meant to include co-administration, as in the case where the material which decreases insulin resistance and the antidepressant/anti-anxiety drug are administered as components of a single composition as well as in the case where the material which decreases insulin resistance and the antidepressant/anti-anxiety drug are administered in separate compositions but at the same time (e.g., as two tablets swallowed simultaneously or as two solutions injected simultaneously). Non-simultaneous administration is meant to include sequential administration (e.g., in the case where the material which decreases insulin resistance is administered before the antidepressant/anti-anxiety drug or in the case where the antidepressant/anti-anxiety drug is administered before the material which decreases insulin resistance). When using sequential administration, it is preferred that the second-to-be-administered drug or material be administered while there is a substantial amount of the first-to-be-administered drug or material present in the subject, for example, by administering the second-to-be-administered drug or material within 3X, 2X, and/or 1X (where X is the first-to-be-administered drug or material's metabolic half-life) of the time at which the first-to-be-administered drug or material was administered. For example, in the case where the antidepressant/anti-anxiety drug is administered before the material which decreases insulin resistance, the material which decreases insulin resistance can be administered to the subject within 4 hours, 3 hours, 2 hours, 1 hour, 30 minutes, 10 minutes, and/or 5 minutes of the time at which the antidepressant/ anti-anxiety drug was administered. In the case where the material which decreases insulin resistance is administered before the antidepressant/anti-anxiety drug, the antidepressant/anti-anxiety drug can be administered to the subject within 4 hours, 3 hours, 2 hours, 1 hour, 30 minutes, 10 minutes, and/or 5 minutes of the time at which the material which decreases insulin resistance was administered.

The aforementioned materials which decrease insulin resistance can be administered alone or in the form of a formulation in combination with suitable pharmaceutical carriers, diluents, or other substances. When administered in the form of a formulation, the glitazone or other material which decreases insulin resistance (as described above) may be used at a concentration of from about 0.1 to about 99.9 weight percent of the formulation.

The pharmaceutical formulation can be in unit dosage form. The unit dosage form can be a capsule or tablet itself, or the appropriate number of any of these. The quantity of glitazone or other material which decreases insulin resistance (“active compound”) in a unit dose of composition may be varied or adjusted from about 0.1 to about 1000 mg or more according to the particular treatment involved.

Compositions (dosage forms) suitable for internal administration typically contain from about 0.1 to about 1000 mg of active compound per unit, such as from about 1 to about 100 mg of active compound per unit and/or from about 2 to about 8 mg of active compound per unit. In these pharmaceutical compositions the active compound will ordinarily be present in an amount of about 0.5-95% by weight based on the total weight of the composition.

The active compound used in the method of the present invention can be administered to treat and/or inhibit the development of Alzheimer's Disease by any means that produces contact of the active compound with its site of action in the human body. As discussed above, the active compounds can be administered alone, but are generally administered with a pharmaceutical carrier selected on the basis of the chosen route of administration and standard pharmaceutical practice. The active compounds can be made up in any suitable form appropriate for the desired use; e.g., oral, parenteral, or topical administration. Examples of parenteral administration are intraventricular, intracerebral, intramuscular, intravenous, intraperitoneal, rectal, and subcutaneous administration.

Suitable formulations include those comprising a therapeutically effective amount of active compound together with a pharmaceutically acceptable diluent or carrier, the composition being adapted for the particular route of administration chosen. By “pharmaceutically acceptable”, it is meant that the carrier, diluent, or excipient is compatible with the active compound in the formulation and not deleterious to the subject being treated.

For the pharmaceutical formulations, any suitable carrier known in the art can be used. In such a formulation, the carrier can be a solid, liquid, or mixture of a solid and a liquid. A solid carrier can be one or more substances which may also act as flavoring agents, lubricants, solubilizers, suspending agents, binders, tablet disintegrating agents, and encapsulating materials.

Tablets for oral administration can contain suitable excipients, such as calcium carbonate, sodium carbonate, lactose, and calcium phosphate, together with disintegrating agents, such as maize, starch, or alginic acid, and/or binding agents, for example, gelatin or acacia, and/or lubricating agents, such as magnesium stearate, stearic acid, or talc. In tablets, the active compound is mixed with a carrier having the necessary binding properties in suitable proportions and compacted in the shape and size desired.

Sterile liquid form formulations include suspensions, emulsions, syrups, and elixirs. The active compound can be dissolved or suspended in a pharmaceutically acceptable carrier, such as sterile water, saline, dextrose solution, sterile organic solvent, or a mixture thereof.

The active compound can be administered orally in solid dosage forms, such as capsules, tablets, and powders, or in liquid dosage forms, such as elixirs, syrups, and suspensions. It can also be administered parenterally in sterile liquid dosage forms. It can also be administered by inhalation in the form of a nasal spray or lung inhaler. It can also be administered topically as an ointment, cream, gel, paste, lotion, solution, spray, aerosol, liposome, or patch. Dosage forms used to administer the active compound usually contain suitable carriers, diluents, preservatives, or other excipients, as described in Remington's Pharmaceutical Sciences, Mack Publishing Company, a standard reference text in the field.

Gelatin capsules can be prepared containing the active compound and powdered carriers, such as lactose, sucrose, mannitol, starch, cellulose derivatives, magnesium stearate, stearic acid, and the like. Similar diluents can be used to make compressed tablets and powders. Both tablets and capsules can be manufactured as sustained release products to provide for continuous release of medication over a period of hours. Compressed tablets can be sugar coated or film coated to mask any unpleasant taste and to protect the tablet from the atmosphere. Additionally or alternatively, tablets can be enteric coated for selective disintegration in the gastrointestinal tract.

Liquid dosage forms for oral administration can contain coloring and flavoring to increase patient acceptance.

For parenteral solutions, water, a suitable oil, saline, aqueous dextrose (glucose), and related sugar solutions and glycols, such as propylene glycol or polyethylene glycols, are suitable carriers. Solutions for parenteral administration contain the active compound, suitable stabilizing agents, and, if necessary, buffer substances. Anti-oxidizing agents, such as sodium bisulfite, sodium sulfite, or ascorbic acid, either alone or combined, are suitable stabilizing agents. Citric acid and its salts and sodium EDTA can also be used. In addition, parenteral solutions can contain preservatives, such as benzalkonium chloride, methyl- or propyl-paraben, and chlorobutanol.

Topical ointments, creams, gels, and pastes contain the active compound and diluents, such as waxes, paraffins, starch, polyethylene glycol, silicones, bentonites, silicic acid, animal and vegetable fats, talc, and zinc oxide, or mixtures of these or other diluents.

Topical solutions and emulsions can, for example, contain in addition to the active compound, customary diluents, such as solvents, dissolving agents and emulsifiers, specific examples of which include water, ethanol, 2-propanol, ethyl carbonate, benzyl alcohol, propylene glycol, oils, glycerol, and fatty acid esters of sorbitol, or mixtures thereof. Compositions for topical dosing may also contain preservatives or anti-oxidizing agents. Solvents having a molecular weight below 200 can be used in the presence of a surface-active agent.

Powders and sprays can contain, along with the active compound, the usual diluents, such as lactose, talc, silicic acid, aluminum hydroxide, calcium silicate, and polyamide powders, or mixtures of these materials. Aerosol sprays can contain the usual propellants. Liposomes can be made from, for example, animal or vegetable fats that form lipid bilayers, and the active compound can be incorporated in such liposomes.

Formulations containing active compounds can be administered through the skin by an appliance, such as a transdermal patch. Patches can be made of a matrix, such as polyacrylamide, and a semipermeable membrane made from a suitable polymer to control the rate at which the material is delivered to the skin. Other suitable transdermal patch formulations and configurations are described, for example, in U.S. Pat. No. 5,296,222 to Petersen et al., and U.S. Pat. No. 5,271,940 to Cleary et al., which are hereby incorporated by reference.

Suitable formulations also include those that further contain (i.e., in addition to a glitazone or other material which decreases insulin resistance and in addition to diluents and other generally non-active components) other active agents. Suitable other active agents include therapeutically or prophylactically effective co-agents for treatment or prevention of Alzheimer's Disease; agents useful in the treatment of type II diabetes; agents useful in the treatment of hyperlipidaemia; agents useful in the treatment of hypertension; agents useful in the treatment of cardiovascular disease; and agents useful in the treatment of eating disorders.

Suitable formulations also include those that do not contain any other active agents.

Suitable formulations also include formulations which contain a glitazone or other material which decreases insulin resistance, but which are substantially free from one, more than one, or all of the following: (i) other agents useful in the treatment of type II diabetes; (ii) other agents useful in the treatment of hyperlipidaemia; (iii) other agents useful in the treatment of hypertension; (iv) other agents useful in the treatment of cardiovascular disease; and (v) other agents useful in the treatment of eating disorders.

Formulations suitable for use in the methods of the present invention, to which formulations the present invention also relates, also include those that further include (i.e., in addition to a glitazone or other material which decreases insulin resistance), one or more antidepressant/anti-anxiety drugs. Suitable antidepressant/anti-anxiety drugs for use in such formulations include selective serotonin reuptake inhibitors (“SSRIs”), such as citalopram (e.g., Celexa™), escitalopram (e.g., Lexapro™), sertraline (e.g., Zoloft™), paroxetine (e.g., Paxil™) and fluoxetine (e.g., Prozac™). Other suitable antidepressant/anti-anxiety drugs that can be used in such formulations include those which inhibit reuptake of brain chemicals other than serotonin, such as venlafaxine (e.g., Effexor™), mirtazapine (e.g., Remeron™) and bupropion (e.g., Wellbutrin™). Still other suitable antidepressant/anti-anxiety drugs that can be used in such formulations include tricyclic antidepressants, such as nortriptyline (e.g., Pamelor™) and desipramine (e.g., Norpramine™). Still other suitable antidepressant/anti-anxiety drugs that can be used in such formulations include anti-anxiety drugs such as buspirone (e.g., Buspa™) and clonazepam (e.g., Klonopin™).

Alzheimer's Disease in a human can be treated by administering glitazone or other material which decreases insulin resistance to treat an existing condition. The glitazone or other material which decreases insulin resistance can also be administered to a subject in anticipation of Alzheimer's Disease, for example, to a patient whose age, lifestyle, genetic disposition, or family history is predictive of the disease.

The dosage of active compound administered will, of course, vary depending upon known factors such as the pharmacodynamic characteristics of the particular active compound and its mode and route of administration; age, health, and weight of the subject; nature and extent of symptoms; kind of concurrent treatment; frequency of treatment; and the effect desired.

In general, the active compound will be administered to a human so that an effective amount is received. An effective amount may conventionally be determined for an individual patient by administering the active compound in increasing doses and observing the effect on the patient, for example, maintenance of memory and cognitive abilities.

Generally, the compound will typically be administered in a manner and a dose to achieve in the human a blood level concentration of active compound which is from about 0.01 to about 100 times, such as from about 0.1 to about 10 times and/or from about 0.5 to about 2 times, the blood level concentration of active compound suitable for the treatment of type II diabetes in humans.

The treatment regimen for many patients afflicted with Alzheimer's Disease may stretch over many years for the remaining life of the patient. Oral dosing may be preferred for patient convenience and tolerance. With oral dosing, suitable dosing can be carried out with one to four oral doses per day, each containing from about 0.5 μg to about 20 mg/kg of body weight, such as from about 5 μg to about 2 mg/kg of body weight and/or from about 5 μg to about 0.1 mg/kg of body weight, of active compound.

Suitable diagnostic criteria for Alzheimer's Disease include those found in standard medical references (e.g., Harrison's Principles of Internal Medicine, 13th ed., McGraw-Hill, Inc., ISBN 0-07-032370-4, pp. 2270-2272 (1994), which is hereby incorporated by reference). These criteria can be used to determine when to begin using the method of the present invention and the frequency and degree of treatment.

A suitable assay protocol for Alzheimer's Disease is found in U.S. Pat. No. 5,686,269 to Nixon, which is hereby incorporated herein by reference, and this assay can be employed in determining the beginning, duration, and end of treatment by the method of the present invention.

Criteria for the clinical diagnosis of probable Alzheimer's Disease can include dementia established by clinical examination and documented by the Mini-Mental State Examination, Blessed Dementia Scale, or some similar examination and confirmed by neuropsychologic tests. Illustratively, such criteria can include: deficits in two or more areas of cognition; progressive worsening of memory and other cognitive functions; no disturbance of consciousness; onset between ages 40 and 90; and/or absence of systemic disorders or other brain diseases that could account for the progressive deficits in memory and cognition. The diagnosis of probable Alzheimer's Disease can be supported by: progressive deterioration of specific cognitive functions such as language (aphasia), motor skills (apraxia), and perception (agnosia); impaired activities of daily living and altered patterns of behavior; family history of similar disorders, particularly if confirmed neuropathologically; and/or laboratory results, for example normal lumbar puncture as evaluated by standard techniques, normal pattern or nonspecific changes in EEG, (such as increased slow-wave activity), and evidence of cerebral atrophy on CT with progression documented by serial observation. Other clinical features consistent with the diagnosis of probable Alzheimer's Disease, after exclusion of causes of dementia other than Alzheimer's Disease, can include: plateaus in the course of progression of the illness; associated symptoms of depression, insomnia, incontinence, delusions, illusions, hallucinations, sexual disorders, weight loss, and catastrophic verbal, emotional, or physical outbursts; other neurologic abnormalities in some patients, especially with more advanced disease and including motor signs, such as increased muscle tone, myoclonus, or gait disorder; seizures in advanced disease; and/or CT normal for age. Features that make the diagnosis of probable Alzheimer's Disease uncertain or unlikely can include: sudden, apoplectic onset; focal neurologic findings such as hemiparesis, sensory loss, visual field deficits, and incoordination early in the course of the illness; and/or seizures or gait disturbances at the onset or very early in the course of the illness. Clinical diagnosis of possible Alzheimer's Disease can be made on the basis of the dementia syndrome, in the absence of other neurologic, psychiatric, or systemic disorders sufficient to cause dementia and in the presence of variations in the onset, presentation, or clinical course. Clinical diagnosis of possible Alzheimer's Disease may also be made in the presence of a second systemic or brain disorder sufficient to produce dementia, such as: familial occurrence; onset before age 65; presence of trisomy 21; and/or coexistence of other relevant conditions, such as Parkinson's Disease.

Other suitable assay protocols for Alzheimer's Disease include those found in U.S. Pat. No. 5,686,476 to May, which is hereby incorporated by reference. This assay can be employed in determining the beginning, duration, and end of treatment by the methods of the present invention.

As discussed above, certain aspects of the present invention relate to methods for treating Alzheimer's Disease and to methods for preventing or inhibiting the development of Alzheimer's Disease in a subject by administering, to the subject, a material which decreases insulin resistance.

The mechanism by which a material that decreases insulin resistance treats Alzheimer's Disease or prevents or inhibits the development of Alzheimer's Disease in the subject is not particularly critical to the practice of the present invention. For example, the mechanism can involve improving HPA axis function or inhibiting HPA axis dysfunction in the subject. Additionally or alternatively, the mechanism can involve inhibiting increases in amyloid β-peptide levels or decreasing amyloid β-peptide levels in the subject. Still additionally or alternatively, the mechanism can involve improving spatial learning or inhibiting spatial learning deficits in the subject. Still additionally or alternatively, the mechanism can involve reducing or inhibiting the development of abnormal anxiety levels and other forms of abnormal stress responses in the subject. Still additionally or alternatively, the mechanism can involve reducing or inhibiting the development of memory impairment in the subject.

As one skilled in the art will appreciate, materials that decrease insulin resistance can be administered to a subject even though the subject is not formally diagnosed with Alzheimer's Disease, for example, to improve HPA axis function or inhibit HPA axis dysfunction in such a subject; to improve spatial learning or inhibit spatial learning deficits in such a subject; to reduce or inhibit the development of abnormal anxiety levels and other forms of abnormal stress responses in such a subject; and/or to reduce or inhibit the development of memory impairment or to improve memory in such a subject. Such subjects can include, for example, those that are predisposed to developing Alzheimer's Disease, as well as those that are not predisposed to developing Alzheimer's Disease; those that suffer from HPA axis dysfunction, as well as those that do not suffer from HPA axis dysfunction; those that suffer from spatial learning deficits, as well as those that do not suffer from spatial learning deficits; those that suffer from type II diabetes, as well as those that do not suffer from type II diabetes; those that suffer from hyperlipidaemia, as well as those that do not suffer from hyperlipidaemia; those that suffer from hypertension, as well as those that do not suffer from hypertension; those that suffer from cardiovascular disease, as well as those that do not suffer from cardiovascular disease; and/or those that suffer from an eating disorder (such as anorexia nervosa, obesity, and anorexia bulimia), as well as those that do not suffer from these or other eating disorders.

In addition to the aforementioned therapeutic and preventative methods, applicant's discovery of the linkage between a subject's insulin resistance and Alzheimer's Disease can be used to detect the presence of Alzheimer's Disease in a subject or the subject's risk for developing Alzheimer's Disease. For example, the present invention, in yet another aspect thereof, relates to a method for detecting Alzheimer's Disease in a subject by assessing insulin resistance in the subject, a high level of insulin resistance being indicative of the presence of Alzheimer's Disease. In another aspect thereof, the present invention relates to a method for assessing a subject's risk for developing Alzheimer's Disease by assessing insulin resistance in the subject, a high level of insulin resistance being indicative of an increased risk for developing Alzheimer's Disease. In still another aspect thereof, the present invention relates to a method for assessing a subject's risk for developing memory impairment, such as memory impairment associated with Alzheimer's Disease, by assessing insulin resistance in the subject, a high level of insulin resistance being indicative of an increased risk for developing memory impairment. In still another aspect thereof, the present invention relates to a method for assessing a subject's risk for developing abnormal stress responses, such as abnormal stress responses characterized by abnormal anxiety levels and/or abnormal stress responses associated with Alzheimer's Disease, by assessing insulin resistance in the subject, a high level of insulin resistance being indicative of an increased risk for developing abnormal stress responses.

The present invention also relates to an article of manufacture. The article of manufacture includes a container; a material which decreases insulin resistance disposed within the container; and a label affixed to the container and/or an insert disposed in the container, wherein the label and/or insert indicates and/or suggests that the material can be used to treat, decrease the risk for development of, and/or inhibit the development of Alzheimer's Disease and/or one or more clinical features of Alzheimer's Disease, such as memory impairment, abnormal stress responses, and the like.

For example, the container can be a plastic or glass bottle, jar, or vial, or it can be a box, such as a cardboard box. In one embodiment, the label can be a paper or plastic sheet that is affixed to the container, for example, by use of an adhesive, tape, shrink wrap plastic, etc. Alternatively or additionally, the label can be affixed to the container by printing the label directly on the material from which the container is made.

Additionally or alternatively to the use of labels, the article can include an insert, such as a printed sheet of paper, generally folded, which is disposed in the container. Illustratively, the container can be a box which contains the insert and the material which decreases insulin resistance. The material which decreases insulin resistance can be disposed directly in the container containing the insert, or it can be disposed in a sub-container, such as a bottle, which, in turn, is disposed in the container. For example, the container can be a box which contains the insert and a plastic bottle or vial, the material which decreases insulin resistance being disposed in the plastic bottle or vial.

The present invention is further illustrated by the following examples.

### EXAMPLES

### Example 1

### Materials and Methods

Male Tg2576 mice and non-transgenic littermates were purchased from Taconic Farms (Germantown, N.Y.). The Tg2576 mice overexpress the 695-isoform of the human amyloid precursor protein containing a double mutation originally identified in a large Swedish kindred (familial AD); spatial learning and memory deficits are evident at 9-11 months of age, prior to the formation of mature amyloid plaques (Kawarabayashi et al., “Age-Dependent Changes in Brain, CSF, and Plasma Amyloid β Protein in the Tg2576 Transgenic Mouse Model of Alzheimer's Disease,” J. Neurosci., 21:372-381 (2001) and Westerman et al., “The Relationship Between Aβ and Memory in the Tg2576 Mouse Model of Alzheimer's Disease.,” J. Neurosci., 22:1858-1867 (2002), which are hereby incorporated by reference). Mice were individually housed in ventilated microisolator cages.

Insulin was administered and glucose was measured using the following procedures. A stock of human insulin (recombinant, 28.7 U.S.P. units/mg; Sigma) was prepared by dissolving 1 mg of powder in 1 ml of dH20 containing 1% glacial acetic acid. A 1:150 dilution in dH20 was used as the injection stock. Mice were administered the insulin solution by intraperitoneal injection. Undiluted whole blood (collected from the tail) was used immediately to measure glucose levels using the Acc.-ChE. Active™ meter (Roche Diagnostics Corporation) or the percent of glycosylated hemoglobin using the Metrika AlcNow™ test kit according to the manufacturers' instructions.

Corticosterone and insulin were measured using the following procedures. For serum preparation, tail bleedings were carried out under a heating lamp, and the blood was collected into MICROTAINER™ Brand Tubes (Becton Dickinson, Franklin Lanes, N.J.). After allowing the blood to clot for 30-40 min, samples were centrifuged for 90 sec at 6000 g and stored at −80° C. until use. Serum corticosterone levels were measured using an enzyme immunoassay kit (Assay Designs, Inc., Ann Arbor, Mich.) with a 1 μl aliquot of each sample diluted 1:100 with assay buffer. The sensitivity of the assay was 26.99 pg/ml. Serum insulin levels were determined using the Ultra Sensitive Rat Insulin ELISA kit (Crystal Chem, Inc., Downers Grove, Ill.) using 5 μl of undiluted sample and mouse insulin standard. The sensitivity of the assay under these conditions was 100 pg/ml. All samples were measured in duplicate for both assays. Plates were read using the Bio-Tek ELx800UV Universal Microplate Reader.

The following supplemented diet was used. Tablets of rosiglitazone maleate (Avandia™, GlaxoSmithKline) were provided by the Creighton University Diabetes Center. The tablets were pulverized and mixed with the AIN-93G purified rodent diet at a final dose of 30 mg/kg of food (Dyets, Inc.). This dose was chosen based on the results of studies in rats using a diet supplemented with rosiglitazone (Diep et al., “Structure, Endothelial Function, Cell Growth, and Inflammation in Blood Vessels of Angiotensin II-Infused Rats: Role of Peroxisome Proliferator-Activated Receptor-γ,” Circulation, 105:2296-2302 (2002), which is hereby incorporated by reference. Unmodified AIN-93G pellets were purchased as the control diet (Dyets, Inc.). The mice were placed on the diets at 9 months of age.

### Example 2

### Insulin Abnormalities in Tq2576 Mice

That male Tg2576 mice exhibit aberrant stress responses and an impaired ability to regulate glucose levels has been previously demonstrated (Pedersen et al., “Aberrant Stress Response Associated with Severe Hypoglycemia in a Transgenic Mouse Model of Alzheimer's Disease,” J. Mol. Neurosci., 13:159-165 (1999) (“Pedersen”), which is hereby incorporated by reference). Furthermore, it has been shown that the mice are glucose intolerant, indicated by higher peak glucose levels relative to wild-type mice following a glucose challenge (Mattson et al., “Cellular and Molecular Mechanisms Underlying Perturbed Energy Metabolism and Neuronal Degeneration in Alzheimer's and Parkinson's Diseases,” Ann. NY Acad. Sci., 893:154-175 (1999), which is hereby incorporated by reference). To provide evidence of insulin resistance in Tg2576 mice and to determine at what age this occurs, the experiments described in this Example 2 were conducted.

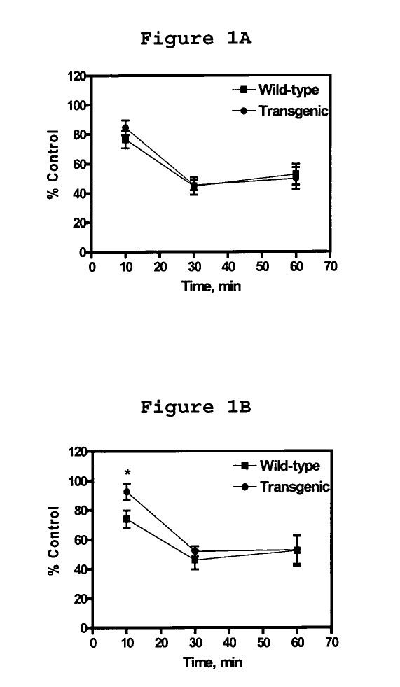

Insulin was administered to non-fasted Tg2576 and wild-type mice at 0.75 U/kg, and blood glucose levels were monitored. At 5 months of age, the magnitude of decline in blood glucose levels following insulin administration was similar in Tg2576 and wild-type mice at all time points examined. This is shown in FIG. 1A. However, as shown in FIG. 1B, at 8 months of age, the Tg2576 mice showed a delay in the insulin-induced decline in blood glucose levels. An overnight fasting of the Tg2576 mice did not alter their responsiveness to insulin (data not shown).

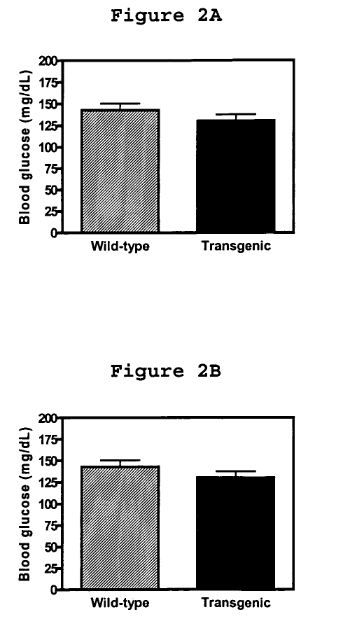

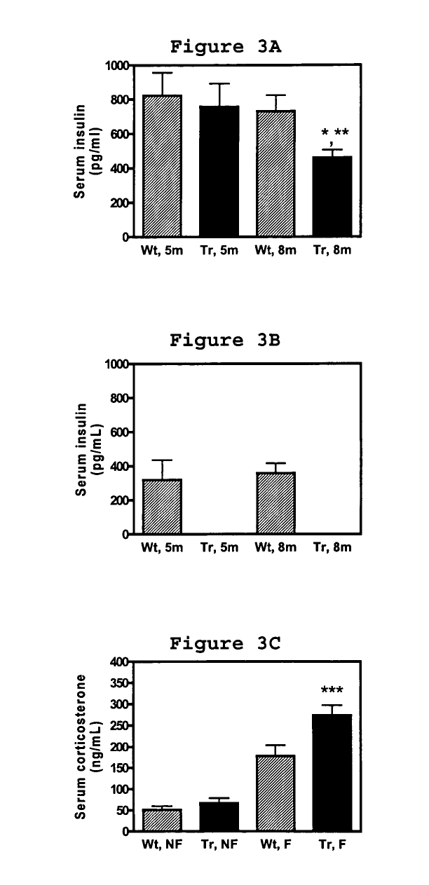

There were no differences in the basal levels of blood glucose or percent glycosylated hemoglobin (a marker of long-term glycemic control) between Tg2576 and wild-type mice, as shown in FIG. 2. The basal serum insulin levels of Tg2576 mice were normal at 5 months of age, but they were significantly reduced by 8 months of age concomitant with the onset of insulin resistance, as shown in FIG. 3A. While an overnight fasting caused the expected decrease in serum insulin levels in wild-type mice, the effect was more pronounced in Tg2576 mice, where the levels dropped to less than 100 pg/ml at both 5 and 8 months of age, as shown in FIG. 3B. There was no difference in basal serum corticosterone levels between the two groups of mice, but there was a greater increase in serum corticosterone levels in Tg2576 mice following an overnight fast, as shown in FIG. 3C, consistent with their abnormal stress responses (Pedersen, which is hereby incorporated by reference).

### Example 3

### Rosiglitazone Normalizes the Stress Responses of Tg2576 Mice

To provide evidence for a relationship between insulin resistance and aberrant stress responses of Tg2576 mice, the animals were subjected to a diet containing the thiazolidinedione, rosiglitazone, in order to increase insulin sensitivity. The thiazolidinediones act as agonists of peroxisome proliferator-activated receptor-γ (“PPARγ”), which is a ligand-inducible transcription factor that regulates glucose and lipid metabolism (Desvergne et al., “Peroxisome Proliferator-Activated Receptors: Nuclear Control of Metabolism,” Endocr. Rev., 20:649-688 (1999), which is hereby incorporated by reference). The agonists of PPARγ attenuate insulin resistance by improving peripheral insulin sensitivity, and such agonists are currently being used clinically in the management of type 2 diabetes (Wagstaff et al., “Rosiglitazone: A Review of its Use in the Management of Type 2 Diabetes Mellitus,” Drugs 62:1805-1837 (2002), which is hereby incorporated by reference). The diet containing rosiglitazone was well tolerated by the mice, as there was no weight loss and the same amount of food was consumed between wild-type mice, transgenics on unsupplemented diet, and transgenics on the rosiglitazone-supplemented diet. More particularly, the following Table 1 summarizes body weights and food consumption for wild-type mice on the regular diet (Wt; n=7), Tg2576 mice on the diet supplemented with rosiglitazone maleate (Tr, AV; n=7), and for Tg2576 mice on the regular diet (Tr, C; n=5). Data are reported as means and standard deviations for the indicated weeks.

TABLE 1

| | Weight of mice (g) | | | Food consumed (g) | | |
|---|---|---|---|---|---|---|
| | Week 1 | Week 4 | Week 7 | Week 1 | Week 4 | Week 7 |
| Wt | 31.6 ± 1.6 | 30.8 ± 1.6 | 31.5 ± 1.5 | 4.2 ± 0.7 | 4.3 ± 0.5 | 4.3 ±0.6 |
| Tr, AV | 26.3 ± 3.2 | 26.2 ± 3.1 | 27.1 ± 3.3 | 4.2 ± 0.6 | 3.8 ± 0.5 | 3.6 ±0.6 |
| Tr, C | 28.6 ± 1.9 | 28.7 ± 2.2 | 28.5 ± 2.0 | 5.3 ± 1.2 | 4.8 ± 0.9 | 5.3 ±1.0 |

Notably, the amount of rosiglitazone-supplemented food consumed by the transgenics decreased over time, which was an expected outcome given that the primary effects of the drug are to increase insulin sensitivity and glucose tolerance. On average, the mice were dosed with approximately 4 mg of drug/day.

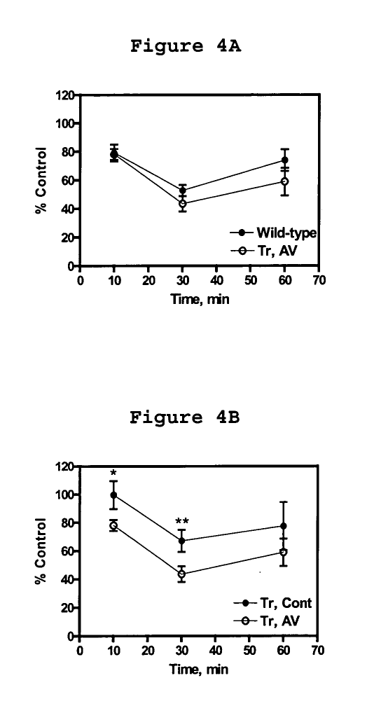

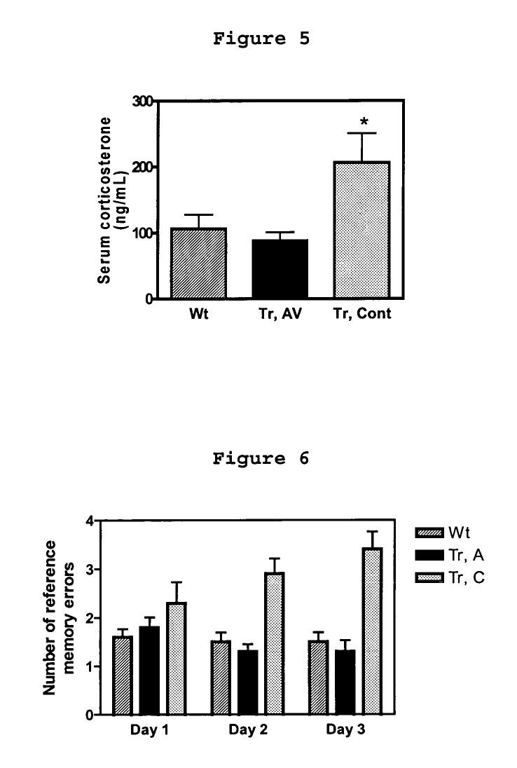

As shown in FIG. 4, the insulin responsiveness of the Tg2576 mice was similar to that of wild-type mice after 4 weeks on the diet. To rule out the possibility that this was simply an aging effect, it was further demonstrated (FIG. 4) that Tg2576 mice on the unsupplemented diet had a much weaker insulin response than Tg2576 mice on the supplemented diet. The study began with 7 mice in each of the three groups. Inherent to the Tg2576 line of mice is a mortality rate of about 20%, and 2 of 7 transgenics on the unsupplemented diet died in the course of the study. None of the 7 transgenics on the rosiglitazone-supplemented diet died, and it was observed that the transgenics on the rosiglitazone-supplemented diet were calmer and easier to handle. Consistent with this observation was a finding that, following an overnight fasting, the serum corticosterone levels increased similarly in wild-type mice and Tg2576 mice on the rosiglitazone-supplemented diet, which were well below the levels reached in Tg2576 mice on the regular diet, as shown in FIG. 5. The serum insulin concentrations of Tg2576 mice remained at approximately 50% of the levels in wild-type mice following rosiglitazone administration (data not shown).

### Example 4

### Effects of Rosiglitazone Maleate (Avandia™) Administration on the Spatial Learning Abilities of Tg2576 Mice

Three groups of mice were included in the study: wild-type mice given a regular diet, transgenic (Tg2576) mice administered the drug Avandia™ in food, and transgenic (Tg2576) mice given a regular diet. After 15 weeks on the drug-supplemented diet, with dosing of approximately 4 mg/kg/day, the Tg2576 mice were subjected to spatial learning testing along with the wild-type and control Tg2576 mice.

The spatial learning abilities and memory function of the different lines of mice were tested using an 8-arm radial maze. Specifically, a Habitest System of Coulbourn Instruments (Allentown, Pa.), a fully automated unit supported by Graphic State Notation 2 software, was used. The software controls the opening and closing of guillotine doors via photocell sensors, allowing the mouse to enter an arm from the central hub. Another photocell sensor is located at the end of each runway, at the pellet trough, to record feeding behavior. For testing, mice were fasted overnight, with access to water ad libitum. An animal was placed in the central hub with all doors closed, and all doors were simultaneously opened when the four arms were baited by the automatic pellet deliverer. Four of the arms were consistently baited with 20 mg food pellets (1, 2, 4, and 7), whereas the other four arms were never baited. As a spatial cue, a small piece of white tape was placed proximal to the floor of the baited arms. The protocol allowed the mice to continue exploring until all four baits in the food troughs were consumed or until 5 minutes had elapsed. This constituted one run. The mice were subjected to 4 runs per day for both training and testing, beginning at 8:00 am each day, with 60-90 min between runs. Training occured over 2 days, immediately followed by testing over 3 days. The number of reference memory errors (entering an arm that is not baited) and the number of working memory errors (entering an arm containing food but one that was previously entered) were recorded.

As shown in FIG. 6, the Tg2576 mice administered the drug (Tr,A) made as many reference memory errors as the wild-type mice (Wt), which in both cases was fewer than the number of reference memory errors made by Tg2576 not given the drug (control Tg2576; Tr,C). The data were analyzed using two-way ANOVA with repeated measures, with Bonferroni post hoc test. There weas a statistically significant difference between the number of errors made between Wt and Tr,C and between Tr,A and Tr,C (p<0.0001); the difference in errors between Wt and Tr,A was not statistically significant. Also, there was not a statistically significant difference between the number of errors made between days for any group.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

1. A method for treating Alzheimer's Disease in a subject, said method comprising:
administering, to the subject, a material which decreases insulin resistance.

2. A method according to claim 1, wherein the material is one which decreases insulin resistance by improving insulin sensitivity.

3. A method according to claim 1, wherein the material is one which decreases insulin resistance by improving insulin sensitivity in peripheral tissues.

4. A method according to claim 1, wherein the material is a PPARγ agonist.

5. A method according to claim 1, wherein the material is a thiazolidinedione.

6. A method according to claim 1, wherein the material is a glitazone or a pharmaceutically acceptable salt, solvate, or adduct thereof.

7. A method according to claim 1, wherein the material is a rosiglitazone or a pharmaceutically acceptable salt, solvate, or adduct thereof.

8. A method according to claim 1, wherein the material is rosiglitazone maleate.

9. A method according to claim 1, wherein the material is a pioglitazone or a pharmaceutically acceptable salt, solvate, or adduct thereof.

10. A method according to claim 1, wherein the material is pioglitazone hydrochloride.

11. A method according to claim 1, wherein said method further comprises:
administering an antidepressant/anti-anxiety drug to the subject.

12. A method according to claim 1, wherein the subject is selected from a mouse, a rat, a dog, and a human.

13. A method according to claim 1, wherein the subject suffers from HPA axis dysfunction.

14. A method according to claim 1, wherein the subject does not suffer from HPA axis dysfunction.

15. A method according to claim 1, wherein the subject suffers from spatial learning deficits.

16. A method according to claim 1, wherein the subject does not suffer from spatial learning deficits.

17. A method according to claim 1, wherein the subject exhibits increased amyloid β-peptide levels.

18. A method according to claim 1, wherein the subject does not exhibit increased amyloid β-peptide levels.

19. A method according to claim 1, wherein the subject suffers from type II diabetes.

20. A method according to claim 1, wherein the subject does not suffer from type II diabetes.

21. A method for preventing or inhibiting the development of Alzheimer's Disease in a subject, said method comprising:
administering, to the subject, a material which decreases insulin resistance.

22. A method according to claim 21, wherein the material is one which decreases insulin resistance by improving insulin sensitivity.

23. A method according to claim 21, wherein the material is one which decreases insulin resistance by improving insulin sensitivity in peripheral tissues.

24. A method according to claim 21, wherein the material is a PPARγ agonist.

25. A method according to claim 21, wherein the material is a thiazolidinedione.

26. A method according to claim 21, wherein the material is a glitazone or a pharmaceutically acceptable salt, solvate, or adduct thereof.

27. A method according to claim 21, wherein the material is a rosiglitazone or a pharmaceutically acceptable salt, solvate, or adduct thereof.

28. A method according to claim 21, wherein the material is rosiglitazone maleate.

29. A method according to claim 21, wherein the material is a pioglitazone or a pharmaceutically acceptable salt, solvate, or adduct thereof.

30. A method according to claim 21, wherein the material is pioglitazone hydrochloride.

31. A method according to claim 21, wherein said method further comprises:
administering an antidepressant/anti-anxiety drug to the subject.

32. A method according to claim 21, wherein the subject is selected from a mouse, a rat, a dog, and a human.

33. A method according to claim 21, wherein the subject suffers from HPA axis dysfunction.

34. A method according to claim 21, wherein the subject does not suffer from HPA axis dysfunction.

35. A method according to claim 21, wherein the subject suffers from spatial learning deficits.

36. A method according to claim 21, wherein the subject does not suffer from spatial learning deficits.

37. A method according to claim 21, wherein the subject exhibits increased amyloid β-peptide levels.

38. A method according to claim 21, wherein the subject does not exhibit increased amyloid β-peptide levels.

39. A method according to claim 21, wherein the subject suffers from type II diabetes.

40. A method according to claim 21, wherein the subject does not suffer from type II diabetes.

41. A composition comprising:
a material which decreases insulin resistance; and
an antidepressant/anti-anxiety drug.

42. A composition according to claim 41, wherein the material which decreases insulin resistance is rosiglitazone maleate.

43. A composition according to claim 41, wherein the material which decreases insulin resistance is pioglitazone hydrochloride.

44. An article of manufacture comprising:
a container;
a material which decreases insulin resistance disposed within the container; and
a label affixed to said container and/or an insert disposed in said container, wherein said label and/or insert indicates and/or suggests that said material can be used to treat, decrease the risk for development of, and/or inhibit the development of Alzheimer's Disease and/or one or more clinical features of Alzheimer's Disease.

45. An article of manufacture according to claim 44, wherein said material comprises a thiazolidinedione.

46. An article of manufacture according to claim 44, wherein said material comprises a glitazone or a pharmaceutically acceptable salt, solvate, or adduct thereof.

47. An article of manufacture according to claim 44, wherein said material comprises a rosiglitazone or a pharmaceutically acceptable salt, solvate, or adduct thereof.

48. An article of manufacture according to claim 44, wherein said material comprises rosiglitazone maleate.

49. An article of manufacture according to claim 44, wherein said material comprises a pioglitazone or a pharmaceutically acceptable salt, solvate, or adduct thereof.

50. An article of manufacture according to claim 44, wherein said material comprises pioglitazone hydrochloride.

51. A method for assessing a test compound's ability to decrease insulin resistance, said method comprising:
administering the test compound to a Tg2576 mouse; and
evaluating whether the Tg2576 mouse exhibits a decrease in insulin resistance.

52. A method for detecting Alzheimer's Disease in a subject or for assessing a subject's risk for developing Alzheimer's Disease, said method comprising:
assessing insulin resistance in the subject, wherein a high level of insulin resistance is indicative of the presence of Alzheimer's Disease or of an increased risk for developing Alzheimer's Disease.

53. A method for assessing a subject's risk for developing memory impairment and/or abnormal stress responses, said method comprising:
assessing insulin resistance in the subject, wherein a high level of insulin resistance is indicative of an increased risk for developing memory impairment and/or abnormal stress responses.

54. A method for improving spatial learning or for inhibiting spatial learning deficits in a subject, said method comprising:
administering, to the subject, a material which decreases insulin resistance.

55. A method for reducing or inhibiting the development of memory impairment or for improving memory in a subject, said method comprising:
administering, to the subject, a material which decreases insulin resistance.
